(19) United States
(12) Patent Application Publication
(10) Pub. No.: US 2025/0124192 A1
(43) Pub. Date: Apr. 17, 2025

(54) MODELING METHOD, MODELING APPARATUS, AND STORAGE MEDIUM

(71) Applicant: Shanghai United Imaging Healthcare Co., Ltd., Shanghai (CN)

(72) Inventor: Jiangfeng Li, Shanghai (CN)

(21) Appl. No.: 18/914,323

(22) Filed: Oct. 14, 2024

(30) Foreign Application Priority Data
Oct. 13, 2023 (CN) 202311332774.X

(51) Int. Cl. G06F 30/25; A61N 5/10; G16H 40/63

(52) U.S. Cl. CPC G06F 30/25; G16H 40/63; A61N 5/1045

(57) ABSTRACT

A modeling method includes: determining a source particle distribution and a leaked particle distribution, determining a first particle ratio between target scattered particles and source particles according to the source particle distribution and the leaked particle distribution, and then determining particle motion parameters of the target scattered particles by sampling according to the first particle ratio and at least one of the leaked particle distribution, an angular distribution of the target scattered particles, and an energy distribution of the target scattered particles, so as to determine a distribution model of the target scattered particles based on the particle motion parameters of the target scattered particles.

### CROSS-REFERENCE TO RELATED APPLICATIONS

This application claims priority to Chinese Patent Application No. 202311332774.X, entitled “MODELING METHOD, MODELING APPARATUS, AND STORAGE MEDIUM” and filed on Oct. 13, 2023, the entire contents of which are incorporated herein by reference.

### TECHNICAL FIELD

The present disclosure relates to the field of radiation delivery technologies, and in particular to a modeling method and apparatus for modeling radiation delivery, and a storage medium.

### BACKGROUND

Radiation dose calculation is an important step during planning of radiation delivery (e.g., radiation therapy, radiation processing, radiation detection, and the like), which determines quality of radiation delivery planning.

Dose calculation relies on modeling of particles in a radiation delivery device. Currently, a motion process of the particles in the radiation delivery device is generally simulated using a Monte Carlo algorithm, to complete modeling of the particles in the radiation delivery device. However, current modeling methods have the problem of low accuracy.

### SUMMARY

In a first aspect, the present disclosure provides a modeling method for modeling radiation delivery, including: determining a source particle distribution and a leaked particle distribution; determining a first particle ratio between target scattered particles and source particles according to the source particle distribution and the leaked particle distribution; determining particle motion parameters of the target scattered particles by sampling according to the first particle ratio and at least one of the leaked particle distribution, an angular distribution of the target scattered particles, and an energy distribution of the target scattered particles; and determining a distribution model of the target scattered particles based on the particle motion parameters of the target scattered particles.

In an embodiment, determining the source particle distribution and the leaked particle distribution includes determining collimator parameters, and determining the source particle distribution and the leaked particle distribution according to the collimator parameters.

In an embodiment, the particle motion parameters include at least one of initial positions, motion directions, or particle energies of the target scattered particles.

In an embodiment, determining particle motion parameters of the target scattered particles by sampling according to the first particle ratio and at least one of the leaked particle distribution, the angular distribution of the target scattered particles, and the energy distribution of the target scattered particles includes: determining a sampling number of the target scattered particles according to the first particle ratio and a total flux of the source particles; and determining the particle motion parameters of the target scattered particles based on the sampling number and at least one of the leaked particle distribution, the angular distribution, and the energy distribution.

In an embodiment, determining the particle motion parameters of the target scattered particles based on the sampling number and at least one of the leaked particle distribution, the angular distribution, and the energy distribution includes sampling the leaked particle distribution based on the sampling number, to obtain initial positions of the target scattered particles.

In an embodiment, determining the particle motion parameters of the target scattered particles based on the sampling number and at least one of the leaked particle distribution, the angular distribution, and the energy distribution includes: sampling motion directions in the angular distribution based on the sampling number, to obtain motion directions of the target scattered particles.

In an embodiment, determining the particle motion parameters of the target scattered particles based on the sampling number and at least one of the leaked particle distribution, the angular distribution, and the energy distribution includes sampling particle energies in the energy distribution based on the sampling number, to obtain particle energies of the target scattered particles.

In an embodiment, determining the first particle ratio between target scattered particles and source particles according to the source particle distribution and the leaked particle distribution includes determining a second particle ratio between leaked particles and the source particles according to the source particle distribution and the leaked particle distribution, and determining the first particle ratio based on the second particle ratio and a preset ratio.

In an embodiment, determining the first particle ratio between target scattered particles and source particles according to the source particle distribution and the leaked particle distribution includes: integrating the leaked particle distribution to obtain a total flux of the leaked particles; integrating the source particle distribution to obtain a total flux of the source particles; and determining the first particle ratio according to the total flux of the leaked particles and the total flux of the source particles.

In an embodiment, determining the first particle ratio according to the total flux of the leaked particles and the total flux of the source particles includes: determining a second particle ratio between the leaked particles and the source particles based on the total flux of the leaked particles and the total flux of the source particles; and determining the first particle ratio according to the second particle ratio and a preset ratio.

In an embodiment, a flux value in the leaked particle distribution is correlated with a sampling probability.

In an embodiment, the source particles comprise particles that are not occluded when emission source emits particles; the leaked particles comprise particles that pass through a collimator; and the scattered particles comprise particles that are scattered by the collimator.

In a second aspect, the present disclosure further provides a modeling method, including: determining a distribution model of target scattered particles based on the collimator scattering source; and adding the distribution model of the target scattered particles to a virtual source model corresponding to a radiation delivery device to obtain a distribution model of particles in the radiation delivery device.

In a third aspect, the present disclosure further provides a computer device, including a memory and a processor. The memory stores a computer program. The processor, when executing the computer program, is configured to perform the modeling method according to any one of the above embodiments.

In a fourth aspect, the present disclosure further provides a non-transitory computer-readable storage medium, having a computer program stored thereon. The computer program, when executed by a processor, causes the processor to perform the modeling method according to any one of the above embodiments.

Details of one or more embodiments of the present disclosure are set forth in the following accompanying drawings and descriptions. Other features, objectives, and advantages of the present disclosure become obvious with reference to the specification, the accompanying drawings, and the claims.

### BRIEF DESCRIPTION OF THE DRAWINGS

In order to more clearly illustrate the technical solutions in embodiments of the present disclosure or the conventional art, the accompanying drawings used in the description of the embodiments or the conventional art will be briefly introduced below. It is apparent that, the accompanying drawings in the following description are only some embodiments of the present disclosure, and other drawings can be obtained by those of ordinary skill in the art from the provided drawings without creative efforts.

FIG. 1 is a diagram of an application environment of a modeling method according to embodiments of the present disclosure;

FIG. 2 is a schematic flowchart of a modeling method according to embodiments of the present disclosure;

FIG. 3 is a schematic diagram of particles according to embodiments of the present disclosure;

FIG. 4 is a schematic flowchart of determining particle motion parameters according to embodiments of the present disclosure;

FIG. 5 is another schematic flowchart of determining particle motion parameters according to embodiments of the present disclosure;

FIG. 6 is a schematic flowchart of determining a first particle ratio according to embodiments of the present disclosure;

FIG. 7 is another schematic flowchart of determining a first particle ratio according to embodiments of the present disclosure;

FIG. 8 is yet another schematic flowchart of determining a first particle ratio according to embodiments of the present disclosure;

FIG. 9 is still another schematic flowchart of determining a first particle ratio according to embodiments of the present disclosure;

FIG. 10 is a schematic flowchart of determining distribution of particles according to embodiments of the present disclosure;

FIG. 11 is a schematic diagram of a principle of a modeling method according to embodiments of the present disclosure;

FIG. 12 is a schematic diagram of a process of a modeling method according to embodiments of the present disclosure;

FIG. 13 is a schematic flowchart of a modeling method according to embodiments of the present disclosure; and

FIG. 14 is a structural block diagram of a modeling apparatus according to embodiments of the present disclosure.

### DETAILED DESCRIPTION

In order to make the objectives, technical solutions, and advantages of the present disclosure clearer, the present disclosure will be further described below in detail with reference to the drawings and embodiments. It should be understood that specific embodiments described herein are intended only to interpret the present disclosure and not intended to limit the present disclosure.

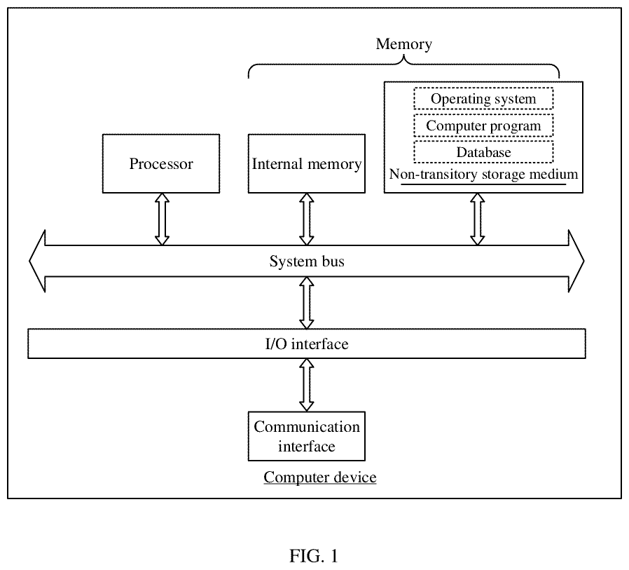

FIG. 1 is a diagram of an application environment of a modeling method according to embodiments of the present disclosure. A computer device is provided in FIG. 1. The computer device may be a server. A diagram of an internal structure thereof may be shown in FIG. 1. The computer device includes a processor, a memory, an input/output (I/O) interface, and a communication interface. The processor, the memory, and the I/O interface are connected by using a system bus. The communication interface is connected to the system bus via the I/O interface. The processor of the computer device is configured to provide computing and control capabilities. The memory of the computer device includes a non-transitory storage medium and an internal memory. The non-transitory storage medium stores an operating system, a computer program, and a database. The internal memory provides an environment for running the operating system and the computer program in the non-transitory storage medium. The database of the computer device is configured to store related data. The I/O interface of the computer device is configured to exchange information between the processor and an external device. The communication interface of the computer device is configured to communicate with an external terminal through a network connection. The computer program is executed by a processor to implement a modeling method.

Those skilled in the art may understand that, the structure shown in FIG. 1 is merely a block diagram of a partial structure related to a solution of the present disclosure, which does not constitute a limitation on the computer device to which the solution of the present disclosure is applied. Specifically, the computer device may include more or fewer components than those shown in the figure, or some components may be combined, or a different component deployment may be used.

This embodiment is illustrated based on an example in which the method is applied to a server. It may be understood that the method is alternatively applicable to a terminal, or is applicable to a system including a terminal and a server and implemented through interaction between a terminal and a server. The terminal may be, but is not limited to, various personal computers, notebook computers, smart phones, and tablet computers. The server may be implemented by a standalone server or a server cluster including a plurality of servers.

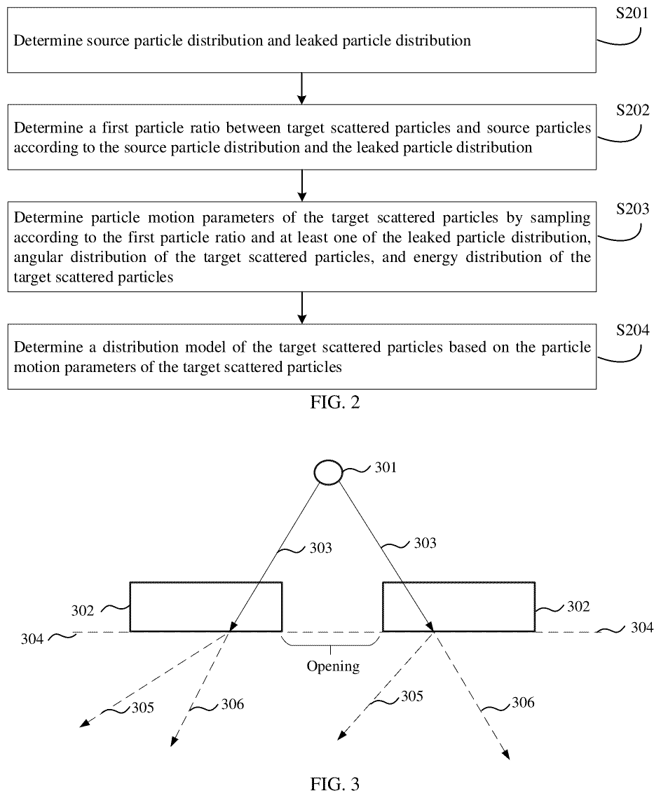

FIG. 2 is a schematic flowchart of a modeling method according to embodiments of the present disclosure. In an exemplary embodiment, as shown in FIG. 2, a modeling method is provided. The description is based on an example in which the method is applied to the computer device in FIG. 1, including steps S201 to S204 below.

In S201, source particle distribution and a leaked particle distribution are determined.

In order to introduce the modeling method in this embodiment more clearly, description is given first with reference to FIG. 3. FIG. 3 is a schematic diagram of particles according to embodiments of the present disclosure. As shown in FIG. 3, an emission source 301 emits particles, and most of the particles may be occluded by collimators 302 and cannot pass through the collimators 302, part of the particles may pass through an opening between the two collimators 302, and a small portion of the particles may pass through the collimators 302. The above particles that are not occluded are collectively called source particles. Taking a position 304 in FIG. 3 as an example, particles arriving at the position 304 are source particles. As can be seen, the source particles include particles passing through the opening and particles passing through the collimators. In the source particles, the particles that pass through the collimators 302 are leaked particles 303. In other words, the leaked particles 303 are the particles in the source particles except those passing through the opening.

The emission source 301 refers to an emission source in a radiation delivery device. The radiation delivery device may include, but is not limited to, treatment heads, radiation detection probes, and other devices that deliver radiation amounts. The collimator 302 may include one or more of a primary collimator, a secondary collimator, and a multi-leaf collimator.

Still referring to FIG. 3, when the leaked particles 303 pass through the collimator, one part of the particles are directly transmitted from the collimator 302 without angular deflection. Such particles are called transmitted particles 306. The other part of the particles are scattered by the collimator 302, and such particles are called scattered particles 305. The scattered particles 305 can illuminate the outside occluded by the collimator 302, thereby increasing a dose outside a radiation field. The applicant wishes to state that although FIG. 3 shows a situation where the scattered particles 305 change motion directions on a lower end face of the collimator 302 (i.e., scattering occurs at the lower end face of the collimator 302), it is only used to illustrate distribution of the particles, and scattering may occur at various parts of the collimator 302, for example, on other end faces of the collimator 302, inside the collimator 302, and the like.

It may be understood that if the opening is smaller, fewer particles pass through the opening, there are more leaked particles 302, and the scattered particles 305 may increase accordingly. Therefore, the applicant realizes that the influence of the scattered particles 305 on dose distribution cannot be ignored.

In this embodiment, the source particle distribution and the leaked particle distribution may be acquired by a computer device first. A manner of acquiring the source particle distribution and the leaked particle distribution is not limited. For example, a pre-stored source particle distribution and a leaked particle distribution may be read from a storage device, the source particle distribution and the leaked particle distribution may be directly measured by using a measuring device such as a detector, the source particle distribution and the leaked particle distribution may be calculated based on various data such as machine parameters and dose measurement values, or the like.

Referring to FIG. 3, the source particle distribution is used to indicate distribution of source particles in the particles emitted from the emission source 301. The leaked particle distribution is used to indicate distribution of the leaked particles 303 in the particles emitted from the emission source 301. The source particle distribution and the leaked particle distribution may include, but are not limited to, any form of a flux map, a histogram, a distribution curve, or a mathematical function model. The source particle distribution and the leaked particle distribution may be in the same form or different forms.

In an embodiment, the source particle distribution includes a source particle flux map, and the leaked particle distribution includes a leaked particle distribution map. Taking the leaked particle flux map as an example, assuming that a coverage area of the collimator is 40 cm×40 cm, the coverage area of the collimator may be divided into 900×900 two-dimensional grids. Each grid can uniquely identify a position in the collimator. For example, Grid 1 represents a position corresponding to a first row and a first column, and Grid 2 represents a position corresponding to a second row and a second column.

Each network corresponds to a flux value ranging from 0 to 1. The flux value is used to represent a proportion of leaked particles in the network region to the particles emitted by the emission source. For example, if the flux value corresponding to Grid 1 is 0, it means that at a position corresponding to Grid 1, the particles emitted by the emission source are all occluded by the collimator. If the flux value corresponding to Grid 1 is 1, it means that at a position corresponding to Grid 1, none of the particles emitted by the emission source are occluded by the collimator. If the flux value corresponding to Grid 1 is 0.1, it means that at the position corresponding to Grid 1, nine-tenths of the particles emitted by the emission source are occluded by the collimator, and one-tenth of the particles are not occluded by the collimator.

It is to be noted that the above only illustrates an alternative form of the leaked particle flux map, and this embodiment is not limited thereto. The same applies to the source particle flux map. Details are not described herein again.

In S202, a first particle ratio between target scattered particles and source particles is determined according to the source particle distribution and the leaked particle distribution.

In this embodiment, after determining the source particle distribution and the leaked particle distribution, the computer device may determine the first particle ratio according to the source particle distribution and the leaked particle distribution. The first particle ratio refers to a ratio of the target scattered particles to the source particles. The target scattered particles refer to scattered particles for modeling in the leaked particles, which may be part or all of the scattered particles corresponding to the leaked particles.

Optionally, the computer device may determine the first particle ratio of the target scattered particles to the source particles according to the source particle distribution, the leaked particle distribution, and a first preset relationship between the source particle distribution, the leaked particle distribution, and the particle ratio of the scattered particles to the source particles. The first preset relationship may be acquired in various manners, as non-limiting examples, which may be calculated according to a physical principle, may be statistically determined based on historical measurement data, may be determined based on user experience, or the like.

For example, it is assumed that the source particle distribution is in Range A and the leaked particle distribution is in Range B. Then, in the first preset relationship, when the source particle distribution is in Range A and the leaked particle distribution is in Range B, a ratio of the scattered particles to the source particles is in Range C, and then the computer device may take any value from Range C as the first particle ratio.

In S203, particle motion parameters of the target scattered particles are determined by sampling according to the first particle ratio and at least one of the leaked particle distribution, an angular distribution of the target scattered particles, and an energy distribution of the target scattered particles.

Since the leaked particles may be reflected inside the collimator, different scattered particles may have different motion directions and particle energies after coming out of the collimator. Therefore, in this embodiment, the computer device is required to determine the particle motion parameters of the target scattered particles by sampling according to the first particle ratio and at least one of the leaked particle distribution, the angular distribution of the target scattered particles, and the energy distribution of the target scattered particles.

The angular distribution is used to indicate a possible angular distribution of the target scattered particles after being emitted from the collimator, which includes a plurality of possible motion directions. The angular distribution may be discrete distribution or continuous distribution. For example, the angular distribution is a distribution function corresponding to a range from 0° to 180°, and 0° to 180° are used to indicate possible motion directions.

The energy distribution is used to indicate possible particle energies of the target scattered particles after being emitted from the collimator, which includes a plurality of possible particle energies. Similarly, the energy distribution may be discrete distribution or continuous distribution. For example, the energy distribution is a distribution function corresponding to E1 to E2, and E1 to E2 are used to indicate possible particle energies. E1 and E2 are two different values, and E2>E1.

The angular distribution or energy distribution of the target scattered particles may be distribution acquired by the computer device from another electronic device, or distribution obtained by the computer device using a preset fitting method, or distribution determined by the computer device in response to a user input operation. This embodiment is not limited thereto.

The particle motion parameters are used to indicate motion of the target scattered particles after being emitted from the collimator. In an exemplary embodiment, the particle motion parameters include at least one of initial positions, motion directions, or particle energies of the target scattered particles. That is, the computer device can determine at least one of initial positions, motion directions, and particle energies of the target scattered particles by sampling according to the first particle ratio and at least one of the leaked particle distribution, the angular distribution of the target scattered particles, and the energy distribution of the target scattered particles.

At least one of the leaked particle distribution, the angular distribution of the target scattered particles, and the energy distribution of the target scattered particles includes one or more of the leaked particle distribution, the angular distribution of the target scattered particles, and the energy distribution of the target scattered particles.

It may be understood that if the computer device makes determination according to the leaked particle distribution and the first particle ratio, initial positions of the target scattered particles may be determined by sampling. If the computer device makes determination according to the angular distribution and the first particle ratio, motion directions of the target scattered particles may be determined by sampling. If the computer device makes determination according to the energy distribution and the first particle ratio, particle energies of the target scattered particles may be determined by sampling.

In the above, the particle motion parameters include at least one of initial positions, motion directions, or particle energies of the target scattered particles. Therefore, flexibility of the particle motion parameters is improved.

It is to be noted that in some embodiments, part or all of the particle motion parameters may be determined by sampling, and part of the particle motion parameters may also be determined by reading pre-stored parameters in the computer device or through experimental measurement.

For example, the computer device may determine the particle motion parameters of the target scattered particles by sampling according to the leaked particle distribution and the first particle ratio, and read pre-stored parameters to determine the particle energies of the target scattered particles.

In S204, a distribution model of the target scattered particles is determined based on the particle motion parameters of the target scattered particles.

In this embodiment, after the computer device determines the particle motion parameters of the target scattered particles, that is, determines the motion of the target scattered particles after being emitted from the collimator, the computer device can determine the distribution model of the target scattered particles based on the particle motion parameters of the target scattered particles. The computer device may determine the distribution model of the target scattered particles based on the particle motion parameters of the target scattered particles by using various algorithms such as a Monte Carlo algorithm, a differential convolution integration algorithm, and a pencil beam algorithm. For example, the computer device may fit the particle motion parameters of the target scattered particles based on the Monte Carlo algorithm by using a preset fitting function, to obtain the distribution model of the target scattered particles. As a non-limiting example, the distribution model of the target scattered particles may show distribution and/or motion of the target scattered particles in space (e.g., near a beam outlet of the radiation delivery device, inside the radiation delivery device, and the like).

Optionally, the computer device may add the distribution model of the target scattered particles to a virtual source model corresponding to the radiation delivery device. The virtual source model may be, for example, a model obtained by modeling particles other than the target scattered particles in the radiation delivery device by using various algorithms such as a Monte Carlo algorithm, a differential convolution integration algorithm, and a pencil beam algorithm. Based on this, simulation of the scattered particles may be added based on the virtual source model in the prior art to obtain a distribution model of the particles in the radiation delivery device, and then the actual distribution of particles emitted from the radiation delivery device can be more accurately simulated, so as to more realistically simulate actual dose distribution, especially in a low-dose region outside a radiation field.

In the above modeling method, the source particle distribution and the leaked particle distribution are determined first, a first particle ratio between target scattered particles and source particles is determined according to the source particle distribution and the leaked particle distribution, and then particle motion parameters of the target scattered particles are determined by sampling according to the first particle ratio and at least one of the leaked particle distribution, an angular distribution of the target scattered particles, and an energy distribution of the target scattered particles, so as to determine a distribution model of the target scattered particles based on the particle motion parameters of the target scattered particles. On the one hand, during the conventional modeling in a manner such as the Monte Carlo algorithm, a scattering effect of the collimator on the particles is not considered, so accuracy is not high. However, in the method provided in this embodiment, since the distribution model of the target scattered particles is determined, the scattering effect of the collimator on the particles is considered, which improves the accuracy of the modeling. On the other hand, during the conventional modeling in a manner such as the Monte Carlo algorithm, since a transport process of particles is simulated by full modeling, there are many simulation events, and calculation time is longer, resulting in low modeling efficiency. In this embodiment, the particle motion parameters of the target scattered particles are directly sampled without simulating the transport process of the particles in the collimator, which reduces the calculation time, thereby improving the modeling efficiency.

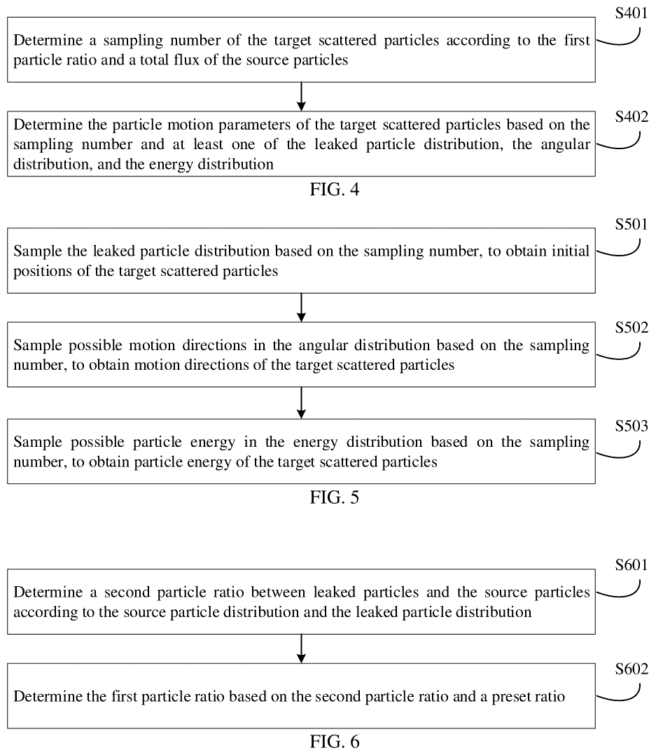

FIG. 4 is a schematic flowchart of determining particle motion parameters according to embodiments of the present disclosure. In an exemplary embodiment, as shown in FIG. 4, S203 includes S401 to S402.

In S401, a sampling number of the target scattered particles is determined according to the first particle ratio and a total flux of the source particles.

In this embodiment, the computer device can determine the total flux of the source particles. The total flux of the source particles is used to indicate a total number of particles emitted from the emission source that are not occluded by the collimator.

Optionally, the computer device may receive the total flux of the source particles sent by another electronic device, the computer device may alternatively determine the total flux of the source particles in response to an input operation, or the computer device may alternatively determine the total flux of the source particles according to a virtual source model of the treatment head.

Further, the computer device may determine the sampling number of the target scattered particles according to the first particle ratio and the total flux of the source particles. Optionally, the computer device may directly take a product of the first particle ratio and the total flux of the source particles as the sampling number of target scattered particles.

In S402, the particle motion parameters of the target scattered particles are determined based on the sampling number and at least one of the leaked particle distribution, the angular distribution, and the energy distribution.

In this embodiment, after determining the sampling number, the computer device may determine the particle motion parameters of certain scattered particles to obtain the particle motion parameters of the target scattered particles based on the sampling number and at least one of the leaked particle distribution, the angular distribution, and the energy distribution, including at least one of initial positions, motion directions, or particle energies of the target scattered particles.

Similarly, at least one of the leaked particle distribution, the angular distribution, and the energy distribution includes one or more of the leaked particle distribution, the angular distribution, and the energy distribution.

For example, the computer device may sample the leaked particle distribution based on the sampling number to obtain the initial positions of the target scattered particles, and may alternatively sample the leaked particle distribution and the angular distribution respectively based on the sampling number to obtain the initial positions and the motion directions of the target scattered particles.

In this embodiment, since the sampling number of the target scattered particles can be determined according to the first particle ratio and the total flux of the source particles, and the particle motion parameters of the target scattered particles can be determined based on the sampling number and at least one of the leaked particle distribution, the angular distribution, and the energy distribution, to obtain at least one of initial positions, motion directions, or particle energies of the target scattered particles Thus, there is no need to simulate the transport process of the scattered particles within the collimator, thereby improving the efficiency of the determination of the particle motion parameters.

FIG. 5 is another schematic flowchart of determining particle motion parameters according to embodiments of the present disclosure. In an exemplary embodiment, as shown in FIG. 5, S402 includes at least one step of S501, S502, and S503.

In S501, the leaked particle distribution is sampled based on the sampling number, to obtain initial positions of the target scattered particles.

In this embodiment, the scattered particles are part of the leaked particles that are scattered by the collimator. Therefore, the initial positions of the target scattered particles can be obtained by sampling the leaked particle distribution

Assuming that the sampling number is N and N is an integer greater than 0, the computer device may sample the leaked particle distribution based on the sampling number to obtain N initial positions corresponding to N target scattered particles.

For example, the leaked particle distribution includes a leaked particle flux map. The computer device may extract positions corresponding to N grids from 900×900 grids as the N initial positions corresponding to the N target scattered particles using methods such as random sampling or uniform distribution sampling. For example, the computer device takes the position corresponding to Grid 2 as the initial position of the target scattered particle 1, the position corresponding to Grid 50 as the initial position of the target scattered particle 2, and the position corresponding to Grid 65 as the initial position of the target scattered particle N.

In S502, possible motion directions in the angular distribution are sampled based on the sampling number, to obtain motion directions of the target scattered particles.

Similarly, the computer device may sample possible motion directions in the angular distribution based on the sampling number, to obtain N motion directions corresponding to the N target scattered particles.

For example, 0° to 180° include all possible motion directions. The computer device may extract 53° from 0° to 180° as the motion direction of the target scattered particle 1 using methods such as random sampling or uniform distribution sampling. For example, the computer device may extract 0° from 0° to 180° as the motion direction of the target scattered particle 2, and so on, and extract 30° from 0° to 180° as the motion direction of the target scattered particle N.

In S503, possible particle energies in the energy distribution is sampled based on the sampling number, to obtain particle energies of the target scattered particles.

Similarly, the computer device may sample possible particle energies in the energy distribution based on the sampling number, to obtain N particle energies of the N target scattered particles.

For example, E1 to E2 includes possible particle energies. The computer device may extract a value from E1 to E2 as the particle energies of the target scattered particle 1 using methods such as random sampling or uniform distribution sampling. For example, the computer device may extract a value from E1 to E2 as the particle energies of the target scattered particle 2, and so on.

It is to be noted that random sampling may be performed in S501 to S503, or sampling may be performed by, but not limited to, stratified sampling, quota sampling, and the like.

In this embodiment, the particle motion parameters of the target scattered particles can be directly sampled, including at least one of the following: sampling the leaked particle distribution based on the sampling number, to obtain initial positions of the target scattered particles; sampling possible motion directions in the angular distribution based on the sampling number, to obtain motion directions of the target scattered particles; and sampling possible particle energies in the energy distribution based on the sampling number, to obtain particle energies of the target scattered particles, thereby improving modeling efficiency.

In an exemplary embodiment, optionally, a flux value in the leaked particle distribution is correlated with a sampling probability.

When there is a need to sample the initial positions of the target scattered particles, since the sampling is performed using the leaked particle distribution and the flux value in the leaked particle distribution can reflect a flux of the leaked particles at corresponding positions, the sampling performed using the leaked particle distribution enables the flux value to be correlated with the sampling probability, so that the sampling process is close to an actual situation, thereby improving the accuracy of sampling.

Sampling probability refers to likelihood of a particular individual or item being selected from a population during a sampling process, for example, likelihood of a position as an initial position of a target scattered particle. Optionally, the flux value in the leaked particle distribution is positively correlated with the sampling probability. In other words, if the flux value in the leaked particle distribution is greater, it is easier to sample the positions as the initial positions of the target scattered particles. A greater flux value in the leaked particle distribution indicates that the flux of the leaked particles at the corresponding positions is greater. Therefore, sampling at positions with more leaked particles can help improve the reliability and accuracy of the particle motion parameters finally obtained.

FIG. 6 is a schematic flowchart of determining a first particle ratio according to embodiments of the present disclosure. In an exemplary embodiment, as shown in FIG. 6, S202 includes S601 to S602.

In S601, a second particle ratio between leaked particles and the source particles is determined according to the source particle distribution and the leaked particle distribution.

In S602, the first particle ratio is determined based on the second particle ratio and a preset ratio.

In this embodiment, the computer device may determine a second particle ratio between leaked particles and the source particles according to the source particle distribution and the leaked particle distribution. For example, the computer device may determine a total flux of the leaked particles according to the source particle distribution, determine a total flux of the source particles according to the leaked particle distribution, and further determine the second particle ratio between the leaked particles and the source particles according to the total flux of the leaked particles and the total flux of the source particles. The computer device may determine the first particle ratio according to the second particle ratio and the preset ratio. The preset ratio may be obtained based on a relationship between the total flux of leaked particles, the total flux of the source particles, and the first particle ratio. The preset ratio may be acquired in various manners, as non-limiting examples, which may be calculated according to a physical principle, may be statistically determined based on historical measurement data, may be determined based on user experience, and the like.

For example, it is assumed that the total flux of the leaked particles is M and the total flux of the source particles is N. In the preset ratio, when the total flux of the leaked particles is M and the total flux of the source particles is N, the first particle ratio between the scattered particles and the source particles is K, and the computer device determines the first particle ratio between the target scattered particles and the source particles to be K. M, N, and K are all numbers greater than 0, and N>M.

In this embodiment, since the second particle ratio between the leaked particles and the source particles can be determined according to the source particle distribution and the leaked particle distribution, the first particle ratio can be efficiently and accurately determined according to the second particle ratio and the preset ratio.

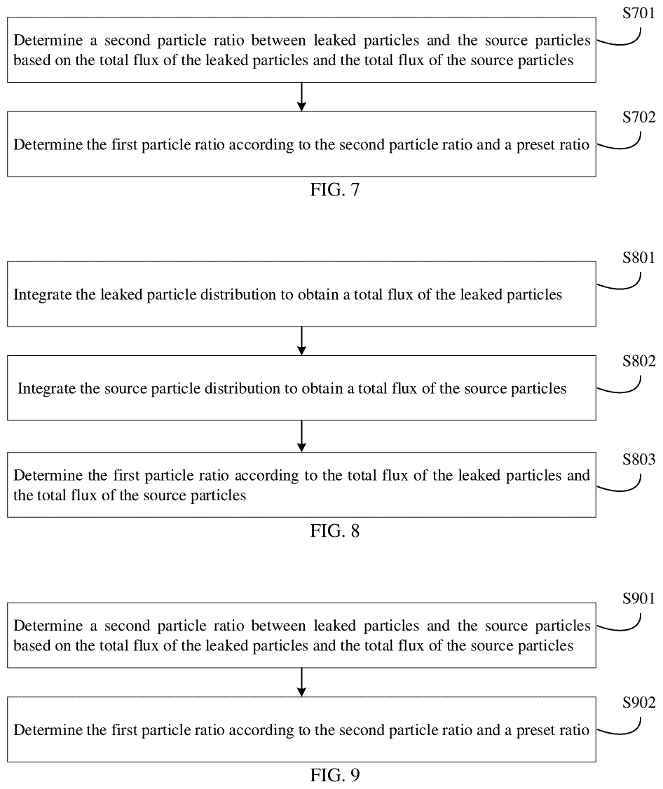

FIG. 7 is another schematic flowchart of determining a first particle ratio according to embodiments of the present disclosure. In an exemplary embodiment, as shown in FIG. 7, S202 includes S701 to S702.

In S701, a second particle ratio between leaked particles and the source particles is determined based on the total flux of the leaked particles and the total flux of the source particles.

In this embodiment, assuming that the total flux of the leaked particles is M and the total flux of the source particles is N, the computer device may take M/N as the second particle ratio. Certainly, the computer device may alternatively perform processing such as rounding on M/N to obtain the second particle ratio.

In S702, the first particle ratio is determined according to the second particle ratio and a preset ratio. The preset ratio is a relationship between the first particle ratio and the second particle ratio.

In this embodiment, the preset ratio may be parameters stored in the computer device in advance. The preset ratio is a relationship between the first particle ratio and the second particle ratio. For example, the preset ratio is obtained based on a proportional relationship between the scattered particles and the transmitted particles in the leaked particles. The preset ratio may also include a proportional relationship between a ratio of the leaked particles to the source particles and a ratio of the transmitted particles to the source particles.

Then, the computer device may determine the first particle ratio according to the second particle ratio and the preset ratio. For example, the computer device determines the second particle ratio to be M/N, and if it is known that the scattered particles in the leaked particles are twice as many as the transmitted particles, the computer device may determine the first particle ratio to be 3M/2N.

In this embodiment, since the preset ratio is a relationship between the first particle ratio and the second particle ratio, the first particle ratio can be determined according to the second particle ratio and the preset ratio after the second particle ratio between the leaked particles and the source particles is determined based on the total flux of the leaked particles and the total flux of the source particles.

FIG. 8 is yet another schematic flowchart of determining a first particle ratio according to embodiments of the present disclosure. In an exemplary embodiment, as shown in FIG. 8, S601 includes S801 to S803.

In S801, the leaked particle distribution is integrated to obtain a total flux of the leaked particles.

In S802, the source particle distribution is integrated to obtain a total flux of the source particles.

In this embodiment, since the leaked particle distribution and the source particle distribution can respectively indicate distribution of the leaked particles and the source particles, the total flux of the leaked particles can be determined by integrating the leaked particle distribution, and the total flux of the source particles can be obtained by integrating the source particle distribution.

In S803, the first particle ratio is determined according to the total flux of the leaked particles and the total flux of the source particles.

In this embodiment, after determining the total flux of the leaked particles and the total flux of the source particles, the computer device may determine the first particle ratio according to the total flux of the leaked particles and the total flux of the source particles.

Optionally, the computer device may determine the first particle ratio according to the total flux of the leaked particles, the total flux of the source particles, and the second preset relationship. The second preset relationship may include a relationship among the total flux of leaked particles, the total flux of the source particles, and the first particle ratio. For example, it is assumed that the total flux of the leaked particles is M and the total flux of the source particles is N. In the second preset relationship, when the total flux of the leaked particles is M and the total flux of the source particles is N, the first particle ratio between the scattered particles and the source particles is K, and the computer device determines the first particle ratio to be K. M, N, and K are all numbers greater than 0, and N>M.

In this embodiment, since the total flux of the leaked particles can be obtained by integrating the leaked particle distribution and the total flux of the source particles can be obtained by integrating the source particle distribution, the first particle ratio can be efficiently and accurately determined according to the total flux of the leaked particles, the total flux of the source particles, and the second preset relationship.

FIG. 9 is another schematic flowchart of determining a first particle ratio according to embodiments of the present disclosure. In an exemplary embodiment, as shown in FIG. 9, S202 includes S901 to S902.

In S901, a second particle ratio between leaked particles and the source particles is determined based on the total flux of the leaked particles and the total flux of the source particles.

In this embodiment, assuming that the total flux of the leaked particles is M and the total flux of the source particles is N, the computer device may take M/N as the second particle ratio. Certainly, the computer device may alternatively perform processing such as rounding on M/N to obtain the second particle ratio.

In S902, the first particle ratio is determined according to the second particle ratio and a preset ratio. The preset ratio is a relationship between the first particle ratio and the second particle ratio.

In this embodiment, the preset ratio may be parameters stored in the computer device in advance. The preset ratio is a relationship between the first particle ratio and the second particle ratio. For example, the preset ratio is obtained based on a proportional relationship between the scattered particles and the transmitted particles in the leaked particles. The preset ratio may also include a proportional relationship between a ratio of the leaked particles to the source particles and a ratio of the transmitted particles to the source particles.

Then, the computer device may determine the first particle ratio according to the second particle ratio and the preset ratio. For example, the computer device determines the second particle ratio to be M/N, and if it is known that the scattered particles in the leaked particles are twice as many as the transmitted particles, the computer device may determine the first particle ratio to be 3M/2N.

In this embodiment, since the preset ratio is a relationship between the first particle ratio and the second particle ratio, the first particle ratio can be determined according to the second particle ratio and the preset ratio after the second particle ratio between the leaked particles and the source particles is determined based on the total flux of the leaked particles and the total flux of the source particles.

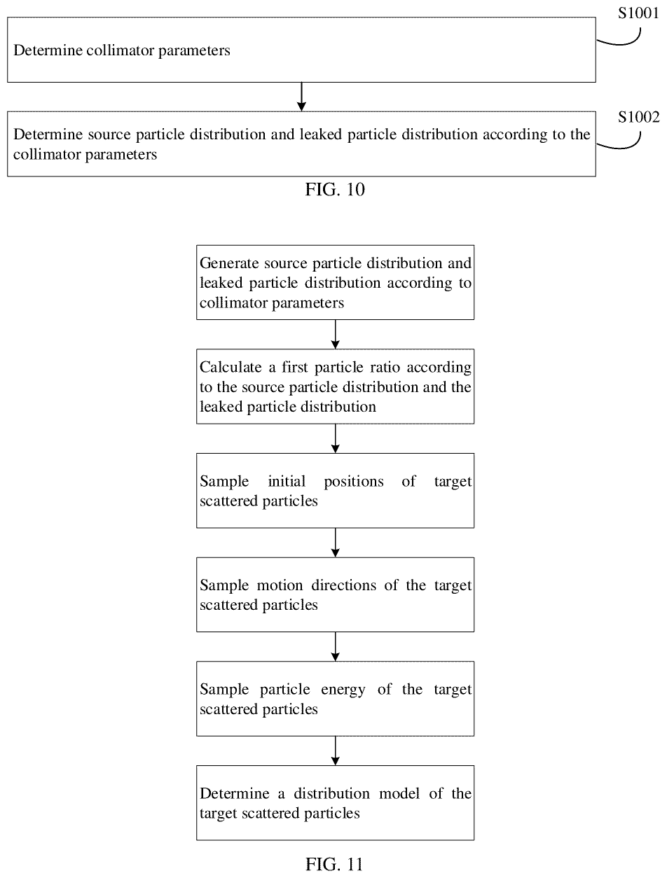

FIG. 10 is a schematic flowchart of determining distribution of particles according to embodiments of the present disclosure. In an exemplary embodiment, as shown in FIG. 10, S201 includes S1001 to S1002.

In S1001, collimator parameters are determined. The collimator parameters include a size and a position of a collimator of a radiation delivery device.

In this embodiment, the radiation delivery device may include, but is not limited to, a treatment head. The collimator may include one or more of a primary collimator, a secondary collimator, and a multi-leaf collimator. As a non-limiting example, the secondary collimator includes, but is not limited to, a jaw, and the multi-leaf collimator includes, but is not limited to, a multi-leaf grating.

The computer device may determine the collimator parameters in response to a user input operation. The computer device may alternatively receive the collimator parameters sent by another electronic device. The collimator parameters may alternatively be parameters stored in the computer device in advance, which is called when the source particle distribution and the leaked particle distribution are required to be determined.

For example, the computer device may determine sizes and positions of the multi-leaf collimator and the jaw.

In S1002, a source particle distribution and a leaked particle distribution are determined according to the collimator parameters.

Further, the computer device may determine the source particle distribution and the leaked particle distribution according to the collimator parameters. Optionally, the computer device may perform projection according to a direction of and a distance between the emission source and the collimator by using the collimator parameters, to determine the source particle distribution and the leaked particle distribution.

In this embodiment, the collimator parameters may include a size and a position of the collimator of the radiation delivery device, more accurate source particle distribution and leaked particle distribution can be determined according to the collimator parameters.

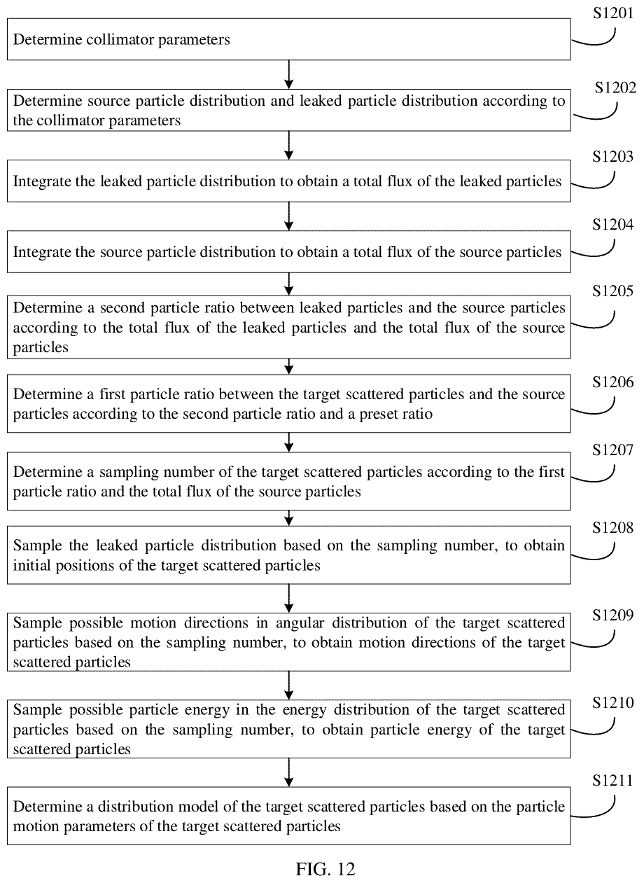

In order to introduce the modeling method in the present disclosure more clearly, description is given herein with reference to FIG. 11 and FIG. 12. FIG. 11 is a schematic diagram of a principle of a modeling method according to embodiments of the present disclosure. As shown in FIG. 11, in this embodiment, the source particle distribution and the leaked particle distribution may be generated according to collimator parameters, and a first particle ratio between the target scattered particles and the source particles may be calculated according to the source particle distribution and the leaked particle distribution. Then, initial positions, motion directions, or particle energies of the target scattered particles may be sampled based on the first particle ratio. Finally, a distribution model of the target scattered particles may be determined based on the initial positions, the motion directions, and the particle energies of the target scattered particles.

FIG. 12 is a schematic diagram of a process of a modeling method according to embodiments of the present disclosure. As shown in FIG. 12, the computer device may perform the method according to the following process.

In S1201, collimator parameters are determined. The collimator parameters include a size and a position of a collimator of a radiation delivery device.

In S1202, a source particle distribution and a leaked particle distribution are determined according to the collimator parameters.

In S1203, the leaked particle distribution is integrated to obtain a total flux of the leaked particles.

In S1204, the source particle distribution is integrated to obtain a total flux of the source particles.

In S1205, a second particle ratio between leaked particles and the source particles is determined according to the total flux of the leaked particles and the total flux of the source particles.

In S1206, a first particle ratio between the target scattered particles and the source particles is determined according to the second particle ratio and a preset ratio. The preset ratio is a relationship between the first particle ratio and the second particle ratio.

In S1207, a sampling number of the target scattered particles is determined according to the first particle ratio and the total flux of the source particles.

In S1208, the leaked particle distribution is sampled based on the sampling number, to obtain initial positions of the target scattered particles.

In S1209, possible motion directions in angular distribution of the target scattered particles are sampled based on the sampling number, to obtain motion directions of the target scattered particles.

In S1210, possible particle energies in the energy distribution of the target scattered particles is sampled based on the sampling number, to obtain particle energies of the target scattered particles. In the above sampling, a flux value in the leaked particle distribution is correlated with a sampling probability.

In S1211, a distribution model of the target scattered particles is determined based on the particle motion parameters of the target scattered particles.

S1201 to S1211 may be obtained with reference to the above embodiments. Details are not described herein again.

It should be understood that, although the steps in the flowcharts as referred to in the embodiments as described above are shown in sequence as indicated by the arrows, the steps are not necessarily performed in the order indicated by the arrows. Unless otherwise clearly specified herein, the steps are performed without any strict sequence limitation, and may be performed in other orders. In addition, at least some steps in the flowcharts as referred to in the embodiments as described above may include a plurality of steps or a plurality of stages, and such steps or stages are not necessarily performed at the same moment, and may be performed at different moments. The steps or stages are not necessarily performed in sequence, and the steps or stages and at least some of other steps or steps or stages of other steps may be performed in turn or alternately.

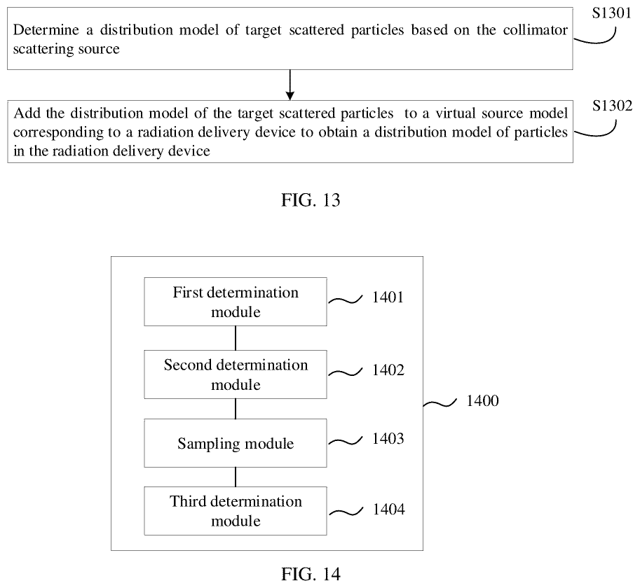

FIG. 13 is a schematic flowchart of a modeling method according to embodiments of the present disclosure. In an exemplary embodiment, as shown in FIG. 13, a modeling method is provided. The description is based on an example in which the method is applied to the computer device in FIG. 1, including steps S1301 to S1302 below.

In S1301, a distribution model of target scattered particles is determined. The distribution model of target scattered particles can be determined based on based on the collimator scattering source. The distribution model can be determined using the method described above.

S1301 may include steps S201 to S204 as described above.

In S1302, the distribution model of the target scattered particles is combined with (e. g., added to) a virtual source model corresponding to a radiation delivery device to obtain a distribution model of particles in the radiation delivery device.

In the embodiment, combining a collimator scattering source with an existing virtual source model can more accurately simulate an actual particle distribution emitted from the treatment head, further improving the accuracy of treatment head modeling.

Further, a dose distribution for dose verification can be obtained based on the distribution model of target scattered particles. For example, the calculated dose distribution is compared to measured beam characteristics from a target radiotherapy machine (e.g., linac) to determine whether the calculated dose satisfies a dose validation criterion (also referred to as a delivery criterion).

Based on the same inventive concept, embodiments of the present disclosure further provide a modeling apparatus configured to implement the modeling method as described above. The implementation solution provided by the apparatus to solve the problem is similar to the implementation solution described in the above method. Therefore, specific limitations in one or more embodiments of the modeling apparatus provided below may be obtained with reference to the limitations on the modeling method above. Details are not described herein.

FIG. 14 is a structural block diagram of a modeling apparatus according to embodiments of the present disclosure. In an exemplary embodiment, as shown in FIG. 14, a modeling apparatus 1400 is provided, including a first determination module 1401, a second determination module 1402, a sampling module 1403, and a third determination module 1404.

The first determination module 1401 is configured to determine a source particle distribution and a leaked particle distribution.

The second determination module 1402 is configured to determine a first particle ratio between target scattered particles and source particles according to the source particle distribution and the leaked particle distribution.

The sampling module 1403 is configured to determine particle motion parameters of the target scattered particles by sampling according to the first particle ratio and at least one of the leaked particle distribution, an angular distribution of the target scattered particles, and an energy distribution of the target scattered particles.

The third determination module 1404 is configured to determine a distribution model of the target scattered particles based on the particle motion parameters of the target scattered particles.

In the above modeling apparatus, a source particle distribution and a leaked particle distribution are determined first, a first particle ratio between target scattered particles and source particles is determined according to the source particle distribution and the leaked particle distribution, and then particle motion parameters of the target scattered particles are determined by sampling according to the first particle ratio and at least one of the leaked particle distribution, an angular distribution of the target scattered particles, and an energy distribution of the target scattered particles, so as to determine a distribution model of the target scattered particles based on the particle motion parameters of the target scattered particles. On the one hand, during the conventional modeling in a manner such as the Monte Carlo algorithm, a scattering effect of the collimator on the particles is not considered, so accuracy is not high. However, in the apparatus provided in this embodiment, since the distribution model of the target scattered particles is determined, the scattering effect of the collimator on the particles is considered, which improves accuracy of the modeling. On the other hand, during the conventional modeling in a manner such as the Monte Carlo algorithm, since a transport process of particles is simulated by full modeling, there are many simulation events, and calculation time is longer, resulting in low modeling efficiency. In this embodiment, the particle motion parameters of the target scattered particles are directly sampled without simulating the transport process of the particles in the collimator, which reduces the calculation time, thereby also improving the modeling efficiency.

Optionally, the particle motion parameters include at least one of initial positions, motion directions, and particle energies.

Optionally, the sampling module 1403 includes:
a first determination unit configured to determine a sampling number of the target scattered particles according to the first particle ratio and a total flux of the source particles; and
a second determination unit configured to determine the particle motion parameters of the target scattered particles based on the sampling number and at least one of the leaked particle distribution, the angular distribution, and the energy distribution.

Optionally, the second determination unit includes:
a first sampling sub-unit configured to sample the leaked particle distribution based on the sampling number, to obtain initial positions of the target scattered particles.

Optionally, the second determination unit includes:
a second sampling sub-unit configured to sample possible motion directions in the angular distribution based on the sampling number, to obtain motion directions of the target scattered particles.

Optionally, the second determination unit includes:
a third sampling sub-unit configured to sample possible particle energies in the energy distribution based on the sampling number, to obtain particle energies of the target scattered particles.

Optionally, the second determination module 1402 includes:
a first determination sub-unit configured to determine a second particle ratio between leaked particles and the source particles according to the source particle distribution and the leaked particle distribution; and
a second determination sub-unit configured to determine the first particle ratio based on the second particle ratio and a preset ratio.

Optionally, the second determination module 1402 includes:
a first integration unit configured to integrate the leaked particle distribution to obtain a total flux of the leaked particles;
a second integration unit configured to integrate the source particle distribution to obtain a total flux of the source particles; and
a third determination unit configured to determine the first particle ratio according to the total flux of the leaked particles and the total flux of the source particles.

Optionally, the third determination unit includes:
a first determination sub-unit configured to determine a second particle ratio between the leaked particles and the source particles based on the total flux of the leaked particles and the total flux of the source particles; and
a second determination sub-unit configured to determine the first particle ratio according to the second particle ratio and a preset ratio, wherein the preset ratio is a relationship between the first particle ratio and the second particle ratio.

Optionally, a flux value in the leaked particle distribution is correlated with a sampling probability.

Optionally, the first determination module 1401 includes:
a fourth determination unit configured to determine collimator parameters, wherein the collimator parameters include a size and a position of a collimator of a radiation delivery device; and
a fifth determination unit configured to determine the source particle distribution and the leaked particle distribution according to the collimator parameters.

The modules in the foregoing image noise reduction processing apparatus may be implemented entirely or partially by software, hardware, or a combination thereof. The above modules may be built in or independent of a processor of a computer device in a hardware form, or may be stored in a memory of the computer device in a software form, to facilitate the processor to invoke and perform operations corresponding to the above modules.

In an exemplary embodiment, a computer device is provided, including a memory and a processor. The memory stores a computer program, and the processor, when executing the computer program, performs the modeling mothed, including:
determining a source particle distribution and a leaked particle distribution;
determining a first particle ratio between target scattered particles and source particles according to the source particle distribution and the leaked particle distribution;
determining particle motion parameters of the target scattered particles by sampling according to the first particle ratio and at least one of the leaked particle distribution, an angular distribution of the target scattered particles, and an energy distribution of the target scattered particles; and
determining a distribution model of the target scattered particles based on the particle motion parameters of the target scattered particles.

In an embodiment, the processor, when executing the computer program, implements the following steps:
determining a source particle distribution and a leaked particle distribution.

In an embodiment, the particle motion parameters include at least one of initial positions, motion directions, and particle energies.

In an embodiment, the processor, when executing the computer program, implements the following steps:
determining a sampling number of the target scattered particles according to the first particle ratio and a total flux of the source particles; and determining the particle motion parameters of the target scattered particles based on the sampling number and at least one of the leaked particle distribution, the angular distribution, and the energy distribution.

In an embodiment, the processor, when executing the computer program, implements the following steps:
sampling the leaked particle distribution based on the sampling number, to obtain initial positions of the target scattered particles.

In an embodiment, the processor, when executing the computer program, implements the following steps:
sampling possible motion directions in the angular distribution based on the sampling number, to obtain motion directions of the target scattered particles.

In an embodiment, the processor, when executing the computer program, implements the following steps:
sampling possible particle energies in the energy distribution based on the sampling number, to obtain particle energies of the target scattered particles.

In an embodiment, the processor, when executing the computer program, implements the following steps:
determining a second particle ratio between leaked particles and the source particles according to the source particle distribution and the leaked particle distribution, and determining the first particle ratio based on the second particle ratio and a preset ratio.

In an embodiment, the processor, when executing the computer program, implements the following steps:
integrating the leaked particle distribution to obtain a total flux of the leaked particles; integrating the source particle distribution to obtain a total flux of the source particles, and determining the first particle ratio according to the total flux of the leaked particles and the total flux of the source particles.

In an embodiment, the processor, when executing the computer program, implements the following steps:
determining a second particle ratio between the leaked particles and the source particles based on the total flux of the leaked particles and the total flux of the source particles, and determining the first particle ratio according to the second particle ratio and a preset ratio.

In an embodiment, the preset ratio is a relationship between the first particle ratio and the second particle ratio.

In an embodiment, a flux value in the leaked particle distribution is correlated with a sampling probability.

In an embodiment, the processor, when executing the computer program, implements the following steps:
determining collimator parameters; and determining the source particle distribution and the leaked particle distribution according to the collimator parameters.

In an embodiment, the collimator parameters include a size and a position of a collimator of a radiation delivery device.

In an embodiment, a non-transitory computer-readable storage medium is provided, having a computer program stored thereon. The computer program, when executed by a processor, causes the processor to perform the modeling method, including:
determining a source particle distribution and a leaked particle distribution;
determining a first particle ratio between target scattered particles and source particles according to the source particle distribution and the leaked particle distribution;
determining particle motion parameters of the target scattered particles by sampling according to the first particle ratio and at least one of the leaked particle distribution, an angular distribution of the target scattered particles, and an energy distribution of the target scattered particles; and
determining a distribution model of the target scattered particles based on the particle motion parameters of the target scattered particles.

In an embodiment, when the computer program is executed by the processor, the following steps are implemented:
determining a source particle distribution and a leaked particle distribution.

In an embodiment, the particle motion parameters include at least one of initial positions, motion directions, and particle energies.

In an embodiment, when the computer program is executed by the processor, the following steps are implemented:
determining a sampling number of the target scattered particles according to the first particle ratio and a total flux of the source particles; and determining the particle motion parameters of the target scattered particles based on the sampling number and at least one of the leaked particle distribution, the angular distribution, and the energy distribution.

In an embodiment, when the computer program is executed by the processor, the following steps are implemented:
sampling the leaked particle distribution based on the sampling number, to obtain initial positions of the target scattered particles.

In an embodiment, when the computer program is executed by the processor, the following steps are implemented:
sampling possible motion directions in the angular distribution based on the sampling number, to obtain motion directions of the target scattered particles.

In an embodiment, when the computer program is executed by the processor, the following steps are implemented:
sampling possible particle energies in the energy distribution based on the sampling number, to obtain particle energies of the target scattered particles.

In an embodiment, when the computer program is executed by the processor, the following steps are implemented:
determining a second particle ratio between leaked particles and the source particles according to the source particle distribution and the leaked particle distribution; and
determining the first particle ratio based on the second particle ratio and a preset ratio.

In an embodiment, when the computer program is executed by the processor, the following steps are implemented:
integrating the leaked particle distribution to obtain a total flux of the leaked particles; integrating the source particle distribution to obtain a total flux of the source particles; and determining the first particle ratio according to the total flux of the leaked particles and the total flux of the source particles.

In an embodiment, when the computer program is executed by the processor, the following steps are implemented:
determining a second particle ratio between the leaked particles and the source particles based on the total flux of the leaked particles and the total flux of the source particles; and determining the first particle ratio according to the second particle ratio and a preset ratio.

In an embodiment, the preset ratio is a relationship between the first particle ratio and the second particle ratio.

In an embodiment, a flux value in the leaked particle distribution is correlated with a sampling probability.

In an embodiment, when the computer program is executed by the processor, the following steps are implemented:
determining collimator parameters; and determining the source particle distribution and the leaked particle distribution according to the collimator parameters.

In an embodiment, the collimator parameters include a size and a position of a collimator of a radiation delivery device.

In an embodiment, a computer program product is provided, including a computer program, wherein when the computer program is executed by a processor, the following steps are implemented:
determining a source particle distribution and a leaked particle distribution;
determining a first particle ratio between target scattered particles and source particles according to the source particle distribution and the leaked particle distribution;
determining particle motion parameters of the target scattered particles by sampling according to the first particle ratio and at least one of the leaked particle distribution, an angular distribution of the target scattered particles, and an energy distribution of the target scattered particles; and
determining a distribution model of the target scattered particles based on the particle motion parameters of the target scattered particles.

In an embodiment, the particle motion parameters include at least one of initial positions, motion directions, and particle energies.

In an embodiment, when the computer program is executed by the processor, the following steps are implemented:
determining a sampling number of the target scattered particles according to the first particle ratio and a total flux of the source particles; and determining the particle motion parameters of the target scattered particles based on the sampling number and at least one of the leaked particle distribution, the angular distribution, and the energy distribution.

In an embodiment, when the computer program is executed by the processor, the following steps are implemented:
sampling the leaked particle distribution based on the sampling number, to obtain initial positions of the target scattered particles.

In an embodiment, when the computer program is executed by the processor, the following steps are implemented:
sampling possible motion directions in the angular distribution based on the sampling number, to obtain motion directions of the target scattered particles.

In an embodiment, when the computer program is executed by the processor, the following steps are implemented:
sampling possible particle energies in the energy distribution based on the sampling number, to obtain particle energies of the target scattered particles.

In an embodiment, when the computer program is executed by the processor, the following steps are implemented:
determining a second particle ratio between leaked particles and the source particles according to the source particle distribution and the leaked particle distribution; and
determining the first particle ratio based on the second particle ratio and a preset ratio.

In an embodiment, when the computer program is executed by the processor, the following steps are implemented:
integrating the leaked particle distribution to obtain a total flux of the leaked particles; integrating the source particle distribution to obtain a total flux of the source particles; and determining the first particle ratio according to the total flux of the leaked particles and the total flux of the source particles.

In an embodiment, when the computer program is executed by the processor, the following steps are implemented:
determining a second particle ratio between the leaked particles and the source particles based on the total flux of the leaked particles and the total flux of the source particles; and determining the first particle ratio according to the second particle ratio and a preset ratio.

In an embodiment, the preset ratio is a relationship between the first particle ratio and the second particle ratio.

In an embodiment, a flux value in the leaked particle distribution is correlated with a sampling probability.

In an embodiment, when the computer program is executed by the processor, the following steps are implemented:
determining collimator parameters; and determining the source particle distribution and the leaked particle distribution according to the collimator parameters.

In an embodiment, the collimator parameters include a size and a position of a collimator of a radiation delivery device.

Those of ordinary skill in the art may understand that some or all procedures in the methods in the foregoing embodiments may be implemented by a computer program instructing related hardware, the computer program may be stored in a non-transitory computer-readable storage medium, and when the computer program is executed, the procedures in the foregoing method embodiments may be implemented. Any reference to the memory, database, or other media used in the embodiments provided in the present disclosure may include at least one of a non-transitory memory and a volatile memory. The non-transitory memory may include a read-only memory (ROM), a magnetic tape, a floppy disk, a flash memory, an optical memory, a high-density embedded non-transitory memory, a resistive random access memory (ReRAM), a magnetoresistive random access memory (MRAM), a ferroelectric random access memory (FRAM), a phase change memory (PCM), a graphene memory, and the like. The volatile memory may include a random access memory (RAM) or an external cache. By way of illustration instead of limitation, the RAM is available in a variety of forms, such as a static random access memory (SRAM) or a dynamic random access memory (DRAM). The database as referred to in the embodiments provided in the present disclosure may include at least one of a relational database and a non-relational database. The non-relational database may include a blockchain-based distributed database, and the like, but is not limited thereto. The processor as referred to in the embodiments provided in the present disclosure may be a general-purpose processor, a central processing unit, a graphics processor, a digital signal processor, a programmable logic device, a data processing logic device based on quantum computing, and the like, but is not limited thereto.

The technical features in the above embodiments may be randomly combined. For a concise description, not all possible combinations of the technical features in the above embodiments are described. However, all the combinations of the technical features are to be considered as falling within the scope described in this specification provided that they do not conflict with each other.

The above embodiments only describe several implementations of the present disclosure, and their description is specific and detailed, but cannot therefore be understood as a limitation on the patent scope of the present disclosure. It should be noted that those of ordinary skill in the art may further make variations and improvements without departing from the conception of the present disclosure, and these all fall within the protection scope of the present disclosure. Therefore, the patent protection scope of the present disclosure should be subject to the appended claims.

What is claimed is:

1. A modeling method for modeling radiation delivery, comprising:
determining a source particle distribution and a leaked particle distribution;
determining a first particle ratio between target scattered particles and source particles according to the source particle distribution and the leaked particle distribution;
determining particle motion parameters of the target scattered particles by sampling according to the first particle ratio and at least one of the leaked particle distribution, an angular distribution of the target scattered particles, and an energy distribution of the target scattered particles; and
determining a distribution model of the target scattered particles based on the particle motion parameters of the target scattered particles.

2. The modeling method according to claim 1, wherein determining the source particle distribution and the leaked particle distribution comprises:
determining collimator parameters; and
determining the source particle distribution and the leaked particle distribution according to the collimator parameters.

3. The modeling method according to claim 1, wherein the particle motion parameters comprise at least one of initial positions, motion directions, or particle energies of the target scattered particles.

4. The modeling method according to claim 3, wherein determining the particle motion parameters of the target scattered particles by sampling according to the first particle ratio and at least one of the leaked particle distribution, the angular distribution of the target scattered particles, and the energy distribution of the target scattered particles comprises:
determining a sampling number of the target scattered particles according to the first particle ratio and a total flux of the source particles; and
determining the particle motion parameters of the target scattered particles based on the sampling number and at least one of the leaked particle distribution, the angular distribution, and the energy distribution.

5. The modeling method according to claim 4, wherein determining the particle motion parameters of the target scattered particles based on the sampling number and at least one of the leaked particle distribution, the angular distribution, and the energy distribution comprises:
sampling the leaked particle distribution based on the sampling number, to obtain initial positions of the target scattered particles.

6. The modeling method according to claim 4, wherein determining the particle motion parameters of the target scattered particles based on the sampling number and at least one of the leaked particle distribution, the angular distribution, and the energy distribution comprises:
sampling motion directions in the angular distribution based on the sampling number, to obtain motion directions of the target scattered particles.

7. The modeling method according to claim 4, wherein determining the particle motion parameters of the target scattered particles based on the sampling number and at least one of the leaked particle distribution, the angular distribution, and the energy distribution comprises:
sampling particle energies in the energy distribution based on the sampling number, to obtain particle energies of the target scattered particles.

8. The modeling method according to claim 1, wherein determining the first particle ratio between target scattered particles and source particles according to the source particle distribution and the leaked particle distribution comprises:
determining a second particle ratio between leaked particles and the source particles according to the source particle distribution and the leaked particle distribution; and
determining the first particle ratio based on the second particle ratio and a preset ratio.

9. The modeling method according to claim 1, wherein determining the first particle ratio between target scattered particles and source particles according to the source particle distribution and the leaked particle distribution comprises:
integrating the leaked particle distribution to obtain a total flux of the leaked particles;
integrating the source particle distribution to obtain a total flux of the source particles; and
determining the first particle ratio according to the total flux of the leaked particles and the total flux of the source particles.

10. The modeling method according to claim 9, wherein determining the first particle ratio according to the total flux of the leaked particles and the total flux of the source particles comprises:
determining a second particle ratio between the leaked particles and the source particles based on the total flux of the leaked particles and the total flux of the source particles; and
determining the first particle ratio according to the second particle ratio and a preset ratio.

11. The modeling method according to claim 4, wherein a flux value in the leaked particle distribution is correlated with a sampling probability.

12. The modeling method according to claim 1, wherein the source particles comprise particles that are not occluded when emission source emits particles;
the leaked particles comprise particles that pass through a collimator; and
the scattered particles comprise particles that are scattered by the collimator.

13. A modeling method for modeling radiation delivery, comprising:
determining a distribution model of target scattered particles; and
combining the distribution model of the target scattered particles with a virtual source model corresponding to a radiation delivery device to obtain a distribution model of particles in the radiation delivery device.

14. A computer device, comprising a memory and a processor, the memory storing a computer program, wherein the processor, when executing the computer program, is configured to perform a modeling method for modeling radiation delivery, the modeling method comprising:
determining a source particle distribution and a leaked particle distribution;
determining a first particle ratio between target scattered particles and source particles according to the source particle distribution and the leaked particle distribution;
determining particle motion parameters of the target scattered particles by sampling according to the first particle ratio and at least one of the leaked particle distribution, an angular distribution of the target scattered particles, and an energy distribution of the target scattered particles; and
determining a distribution model of the target scattered particles based on the particle motion parameters of the target scattered particles.

15. The computer device according to claim 14, wherein the processor, when executing the computer program, is configured to:
determine collimator parameters; and
determine the source particle distribution and the leaked particle distribution according to the collimator parameters.

16. The computer device according to claim 14, wherein the particle motion parameters comprise at least one of initial positions, motion directions, or particle energies of the target scattered particles, and the processor, when executing the computer program, is configured to:
determine a sampling number of the target scattered particles according to the first particle ratio and a total flux of the source particles; and
determine the particle motion parameters of the target scattered particles based on the sampling number and at least one of the leaked particle distribution, the angular distribution, and the energy distribution.

17. The computer device according to claim 16, wherein the processor, when executing the computer program, is configured to:
sample the leaked particle distribution based on the sampling number, to obtain initial positions of the target scattered particles.

18. The computer device according to claim 16, wherein the processor, when executing the computer program, is configured to:
sample possible motion directions in the angular distribution based on the sampling number, to obtain motion directions of the target scattered particles.

19. The computer device according to claim 16, wherein the processor, when executing the computer program, is configured to:
sample possible particle energies in the energy distribution based on the sampling number, to obtain particle energies of the target scattered particles.

20. A non-transitory computer-readable storage medium, having a computer program stored thereon, wherein when the computer program, when executed by a processor, causes the processor to perform the modeling method for modeling radiation delivery according to claim 1.
